(19) United States
(12) Patent Application Publication
(10) Pub. No.: US 2007/0233258 A1
(43) Pub. Date: Oct. 4, 2007

(54) Vertebroplasty- device and method

(75) Inventors: Hugh D. Hestad, Edina, MN (US); Jack Maertens, Chaska, MN (US); Bruce Robie, Glen Rock, NJ (US); Robert Garryl Hudgins, Burnsville, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/364,455

(22) Filed: Feb. 28, 2006

(51) Int. Cl. A61F 2/44

(52) U.S. Cl. 623/17.12

(57) ABSTRACT

A device for treating a vertebral body may include a hollow device made from a permeable fabric and having at least one opening for introducing bone treatment material into the device. Introduction of the bone treatment material may cause the bone treatment material to permeate through the permeable fabric of the device.

### TECHNICAL FIELD

The present invention generally relates to treating a fractured bone. More particularly, the present invention relates to a device and method for treating damage in vertebral bodies.

### BACKGROUND

The human spine consists of a complex set of interrelated anatomic elements including a set of bones called vertebral bodies. Aging and disease, among other conditions, negatively impact the spine. Osteoporosis and meta-static disease reduce the structural integrity of the vertebral bodies, predisposing them to fracture. Vertebral fracture can result in loss of vertebral height which in turn can exacerbate neurological conditions or lead to other symptoms.

Generally, fractures and loss of height, if not treated, result in a cascade of undesirable injuries. These conditions often result in back pain. Vertebroplasty is an attempt towards stabilizing these fractures and to alleviate this source of pain.

U.S. Pat. Nos. 5,549,679 and 5,571,189 to Kuslich and U.S. Patent Publication No. 2004/0073308 to Kuslich et al. describe devices and methods for stabilizing spinal segments by first accessing and boring into the damaged tissue or bone and reaming out the damaged and/or diseased area. Next, a porous fabric bag positioned over an inflation balloon is inserted into the reamed out section and the balloon inflated to compact the cavity wall. The bag is then filled with fill material under pressure either with or without leaving the balloon in place. These methods require the step of inflating the balloon within the bag prior to filling the bag with fill material under pressure.

U.S. Pat. No. 6,740,093 and U.S. Patent Publication No. 2004/0215344 to Hochschuel et al. disclose a container which is permanently implanted to stabilize the vertebral body or to restore height to the vertebral body. In one embodiment the container is porous to the bone filler material, and in another embodiment the container is impermeable to the bone filler material. In each instance, the container controls and regulates the delivery of bone filler material into the vertebral body. The container may be flexible and conformable to the cavity or it may be of a fixed shape which conforms to the cavity shape when deployed. The bone filler may be injected into the container until the cavity is completely filled and thereby stabilizing the vertebral body. Alternately, the vertebral body is stabilized by injected bone filler to displace the end plates of the vertebral body in a hydraulic jacking effect.

U.S. Pat. Nos. 5,108,404 and 4,969,888 to Scholten et al. describe systems for fixing osteoporotic bone using an inflatable balloon to compact the bone and form a cavity into which bone cement is introduced under pressure after the balloon is removed. The use of fluoroscopy is necessary to monitor the introduction of the bone cement for guarding against cement leakage through fissures in the bone. In spite of precautions, cement leakage is known to occur.

U.S. Pat. No. 5,972,015 to Scribner et al. describes a system for deploying a catheter tube into the interior of a vertebra and expanding a specially configured nonporous balloon therewithin to compact cancellous bone and form a cavity. The cavity thus formed is next filled with bone cement under pressure which, as previously discussed, is known to leak out of the cavity.

Bone treatment material is often delivered to the treatment site under pressure. Even under controlled conditions and extreme caution, some bone treatment material could enter the blood vessels and venous cavities resulting in the formation of emboli. The flowing blood caries away these emboli and can result in blocked blood vessels in the heart, brain, and other areas. This can result in serious injury, including paralysis and death.

Some of the prior art suggests the use of an impermeable balloon, bag, etc. for confining the bone treatment material to the treatment site and thereby preventing leakage. However, the use of such impermeable containers will also impede the penetration of the bone treatment material into the voids and fissures at the treatment site.

Accordingly, there is a continuing need for improved devices and methods for treating damaged vertebral bodies while minimizing risks to the patient.

### SUMMARY

The present invention discloses a device and a method for treating vertebral bodies.

One embodiment of the present invention includes a device made from a permeable fabric with an opening adapted for introducing bone treatment material into the device. The permeable fabric is flexible and collapsible, weaved from a fiber which is metallic, non-metallic, or a combination thereof. The weave density of the fabric may be modifiable such that the fabric may have one or more regions of predetermined and distinct permeability over the surface of the device whereby the permeability at a location on the surface of the device may be relatively more or less than the permeability at another location on the surface of the device.

In a method according to an embodiment of the present invention, the device is delivered to the treatment site within the vertebral body. Next, bone treatment material is introduced into the device under pressure, causing the device to expand. Bone at the treatment site gets compacted, and the bone treatment material permeates through the surface of the device, entering voids and fissures at the treatment site.

For those skilled in the art, a more complete understanding of the present invention, and alternative embodiments, will become apparent from the following drawings, their detailed description, and the appended claims. As will be realized, the embodiments may be modified in various aspects without departing from the scope and spirit of the present invention. Accordingly, the drawings and detailed description are to be regarded as illustrative in nature and not restrictive.

### BRIEF DESCRIPTION OF THE DRAWINGS

FIG. 1 is a perspective view of a device in accordance with an embodiment of the present invention for treating a vertebral body;

FIG. 2A is a plan view of a vertebrae showing a treatment site;

FIG. 2B is a plan view of the vertebrae of FIG. 2A showing delivery of the device of the present invention to the treatment site in the vertebrae;

FIG. 2C is a plan view of the vertebrae of FIG. 2B showing the device of the present invention in a collapsed state;

FIG. 2D is a plan view of the vertebrae of FIG. 2C showing the device of the present invention in an expanded state;

FIG. 3A is a side elevational view of a vertebrae showing a treatment site;

FIG. 3B is a side elevational view of the vertebrae of FIG. 3A showing delivery of the device of the present invention to the treatment site in the vertebrae;

FIG. 3C is a side elevational view of the vertebrae of FIG. 3B showing the device of the present invention in a collapsed state; and

FIG. 3D is a side elevational view of the vertebrae of FIG. 3C showing the device of the present invention in an expanded state.

### DETAILED DESCRIPTION

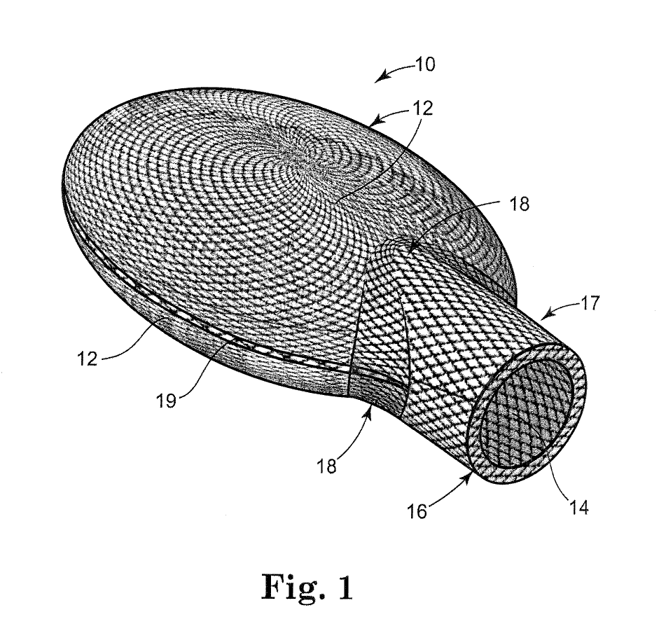

A device 10 for treating vertebral bodies in accordance with an embodiment of the present invention is shown in FIG. 1. Device 10 includes permeable body 12, interior cavity 14, opening 16, port 17, and neck 18. Port 17 provides fluid communication between interior cavity 14 and opening 16, and is used for introducing bone treatment material into interior cavity 14 of device 10. In one embodiment of the present invention, device 10 may be a circular- or elliptical-shaped bag-like hollow disc such as that shown in FIG. 1. Alternately, device 10 may have a different geometric shape such as a cylinder, a sphere, etc. In another embodiment, device 10 may be custom-shaped for the treatment site prior to its delivery into the vertebral body. In yet another embodiment, device 10 may adapt and conform to the shape and size of the treatment site upon delivery.

In accordance with an embodiment of the present invention, permeable body 12 may be formed from a permeable fabric. In one embodiment this fabric is formed by weaving fibers of one type of material or material with other components. The permeability of the woven fabric may in part depend on the density of the weave and/or the nature of the weave. For instance, the fabric may have a multi-dimensional weave. Moreover, the fabric may be one in which the fibers form fully interconnected interstitial spaces. A fabric having fully interconnected interstitial spaces is one in which all the spaces formed by the weave pattern are interconnected with one another. In other words, each space formed by the weave pattern is directly or indirectly connected to every other space formed by the weave pattern.

One example of a three dimensional weave may be a fabric with a honeycomb weave with a three-dimensional cell-like structure in which long floats form the periphery of the cells. An open space fully circumscribes each individual fiber, and adjacent spaces formed by the weaving of the fibers are off-set from one another. The interlacing is progressively tightened, towards the cell center, with the tightest interlacing occurring at the center of the cell. This weave pattern creates a structure of hollow pockets between raised portions, similar to a waffle. The face and the back of the fabric look alike, the midpoint of the cell on one side serving as the outer corner on the other side, i.e., the high point on one side of the fabric is the low point on the other side.

It will be apparent to one skilled in the art that the weave density, and therefore the cross-sectional area of the flow-path between adjacent spaces, may be affected by the density of the fibers during the weaving process and also the degree of offset of the stacked spaces. Thus, the density of the fibers forming the weave, by impacting the cross-sectional area of the flow-path between adjacent spaces, may contribute to the permeability of the fabric. Other factors that contribute to the permeability include the type of fabric and the type of material that is being passed through the weave.

Alternate embodiments of permeable body 12 may include a permeable fabric with one or more regions of predetermined and distinct weave density. In another embodiment, permeable body 12 may include a permeable fabric with continuously varying weave density. Other embodiments may include a fabric with variable permeability and/or pressure drop. Pressure drop is defined as the drop in fluid pressure across the thickness of the fabric when a material is forced through it. Further embodiments may include a permeable fabric with one or more regions of predetermined and distinct permeability. In such embodiments, the variable permeability and/or pressure drop may be achieved by parameters such as weave density, form and/or shape of the weave, nature of the fibers, etc. In one such embodiment, adjacent sections or locations of the permeable fabric may have slightly and/or vastly different weave density or permeability. In further embodiments, the pressure drop across the thickness of the fabric may be altered depending on the permeability and type of weave. Additional embodiments may include a permeable fabric with directionally variable expansion characteristics. Other embodiments may include a permeable fabric with one or more regions of predetermined and distinct expansion rates. The expansion characteristics may be determined, in part, by the weave density and the weave pattern and type. As can be seen, several alternative embodiments of the fabric are possible wherein the permeability can be selected by varying structural parameters such as size, shape, pattern, etc.

Further embodiments of permeable body 12 may include a fabric to which a material has been applied in order to alter the permeability of the fabric. An alternate embodiment may be one in which the permeability at different sections of permeable body 12 is changed by applying different types and/or different quantities of the material to the different sections of permeable body 12. The material may be of a type which penetrates into the fabric and affects its permeability by altering the cross-sectional area of the flow path. Alternately, the material may be applied to the surface of permeable body 12 without penetrating into the fabric. In further embodiments the material may affect how the fiber or other material used to form the weave interacts with the bone treatment or other material passing through the weave. One example of a material that may be used is urethane, which is known to be bio-compatible.

Under one embodiment of the present invention, the permeable fabric may require a large pressure drop for permeating the bone treatment material through permeable body 12. Such permeable fabric may allow the bone treatment material to permeate through permeable body 12 in a relatively slow, and therefore in a relatively more controlled, manner. As is well known in the art, bone treatment material may include a flowable in-situ curable bio-compatible material. Some examples of such bone treatment materials may include polymethylmethacrylate (PMMA), bisphenol-A-glycidyidimethacrylate (BIS-GMA) materials such as CORTOSS™ and dental composites, gypsum-based composites, polyurethane, etc.

An alternate embodiment of the present invention may further include band 19 that is substantially impermeable relative to the permeability of permeable body 12. The relative impermeability along band 19 of device 10 may prevent, or substantially minimize, leakage of the bone treatment material from band 19. As may be appreciated, band 19 may fully circumscribe device 10 or may be disposed in any desired pattern or template on any desired position on device 10 for achieving a desired result. Band 19 may be narrow, broad, thick, thin, or continuously variable as desired.

In another embodiment of the present invention, port 17 and neck 18 of device 10 may be substantially impermeable relative to the permeability of permeable body 12. In an embodiment of the present invention, permeability of port 17, neck 18, and band 19 may be similar or different from one another. The relative impermeability along band 19, port 17, and neck 18 of device 10 may prevent, or substantially minimize, leakage of the bone treatment material from these sections of device 10. In one such design of device 10, a substantial portion of the bone treatment material introduced into interior cavity 14 of device 10 may be directed to permeate through an upper and lower side of permeable body 12. In further embodiments, the bone treatment material may permeate in a variety of narrow or large sections on the top, bottom, or sides of device 10. In an alternate embodiment of the present invention, the permeability along port 17, neck 18, and band 19 of device 10 may be the same or only slightly different than the permeability of permeable body 12.

In an embodiment of the present invention, port 17 leading to interior cavity 14 of device 10 may removeably encase the distal end of a cannula thereby establishing fluid communication between interior cavity 14 of device 10 and the proximal end of the cannula, the proximal end of the cannula being located outside the patient's body. Means such as a drawstring on port 17 may be used for enabling port 17 to securely encase the distal end of the cannula while the bone treatment material is introduced into interior cavity 14 of device 10. Alternate means such as an elastic band, a hose clamp, shrink wrap, etc., may also be used, either independently or in combination, for enabling port 17 to securely encase the distal end of the cannula. Such securing means would enable port 17 to grip the distal end of the cannula with sufficient tightness so as to prevent, or minimize, leakage of the bone treatment material.

Device 10, and in particular permeable body 12, may be formed of a permeable fabric that is flexible and/or collapsible such that device 10 may be manipulated easily for delivery to the treatment site. Device 10 may be delivered to the treatment site through a portal or a cannula-like device such as a catheter, a stylet, or the like. Device 10 may be further capable of regaining its normal shape or close to its normal shape when extracted from the delivery device by shape memory or by introduction of the bone treatment material under pressure into cavity 14 of device 10.

Introduction of the bone treatment material under pressure into cavity 14 of device 10, through opening 16, may cause device 10 to bulge and compact the bone at the treatment site. Continued introduction through opening 16 of the bone treatment material under pressure into cavity 14 of device 10 may enable the bone treatment material to permeate out of device 10 through permeable body 12.

The bulging of device 10 may provide an increase in the contact area between permeable body 12 and the vertebral bone surface at the treatment site. Bone treatment material permeating out of device 10 through permeable body 12 may enter voids and fissures in the vertebral body at the treatment site and aid in strengthening the vertebral body. Additionally, the permeating bone treatment material may substantially encase the fabric of permeable body 12 and incorporate the permeable fabric as part of the final repair structure. When the bone treatment material cures, the encased fabric of permeable body 12 may provide additional structural integrity and strength to the treated vertebral body. This configuration may be similar to the use of a re-bar and/or a mesh in strengthening concrete structures.

Certain embodiments of the present invention may include a back-flow prevention means, such as a flap or damper, within device 10 to prevent leakage of the bone treatment material from opening 16 during and/or after filling device 10. The back-flow prevention means permits the unhindered flow of the bone treatment material into device 10 and impedes any flow out of opening 16.

In one embodiment, permeable body 12 of device 10 may be formed of any type of immunologically inert fabric compatible with the environment within a mammalian body, and in particular, within a vertebral body. As is well known to one skilled in the art, an immunologically inert fabric may inhibit a significant response by the immune system when implanted into a subject.

In another embodiment of the present invention, permeable body 12 may comprise a woven permeable fabric formed from one or more fibers from the group consisting of: polymeric material such as an aramid (e.g., Kevlar™, Nomex™, Twaron™, etc.), polyester such as Dacron™, an ultra high molecular weight highly oriented and highly crystalline polyethylene (e.g., Dyneema™, Spectra™ 900, Spectra™ 1000, etc.), nylon, silk, elastin, elastomeric (e.g., polyurethane, thermoplastic elastomer, etc.), cellulose, polytetrafluoroethylene (PTFE, e.g., fused, expanded, etc.), polyacrylonitrile, and the like. In an alternate embodiment, permeable body 12 may comprise a fabric formed from a metallic fiber such as: nitinol, stainless steel (e.g., heat-treated 17-7 PH™ stainless steel), or the like. In other embodiments, device 10 may be made using a combination of materials, such as, for example, a combination of a polymeric fiber and a metallic material. In yet another embodiment, permeable body 12 may be made from a composite of any one or more of the aforementioned materials. An embodiment of permeable body 12 may comprise one or more layers of one or more permeable fabric.

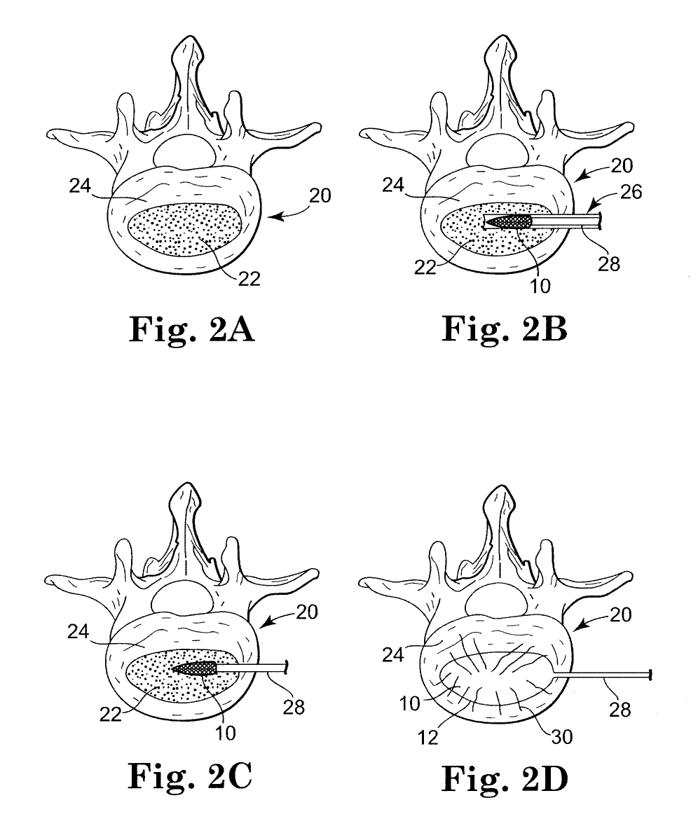

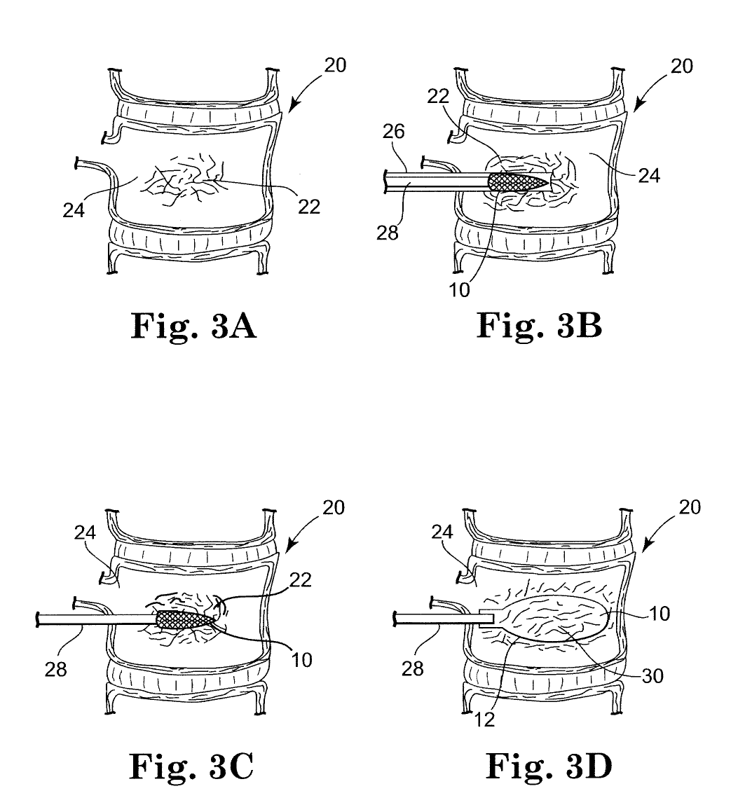

Next, FIGS. 2A-2D and 3A-3D will be discussed in terms of a method for treating a vertebral body in accordance with an embodiment of the present invention. FIGS. 2A-2D are a plan view and FIGS. 3A-3D are an elevation view of selected steps in the treatment process.

It will be apparent to one skilled in the art, that the approach, path or the location from which entry is made into the vertebral body as shown in FIGS. 2A-2D and 3A-3D are for illustration purposes only Several alternative approach paths are well known in the art.

FIGS. 2A and 3A illustrate a treatment site 22 within bone 24 of vertebral body 20. Using means well known in the art, devices such as delivery device 26 may be used for positioning device 10 at treatment site 22 as shown in FIGS. 2B and 3B.

Fluoroscopy, imaging, etc., may be used for monitoring the placement of device 10 at treatment site 22. After device 10 has been positioned at treatment site 22, delivery device 26 may be removed from vertebral body 20 leaving device 10 exposed as illustrated in FIGS. 2C and 3C. Alternately, delivery device 26 may be used to expose device 10 within treatment site 22, as, for instance, by withdrawing distal end of delivery device 26 over and past the location where port 17 of device 10 encases the distal end of cannula 28.

Next, means such as cannula 28, also well known in the art, may be used for introducing the bone treatment material under pressure into interior cavity 14 of device 10. Means for introducing bone treatment material into interior cavity 14 of device 10 under pressure may include a syringe, a pumping mechanism, and the like. Furthermore, means well known in the art, such as fluoroscopy, imaging, etc., may be used for monitoring the introduction of the bone treatment material at treatment site 22. As shown in FIGS. 2D and 3D, the introduction of bone treatment material under pressure may cause device 10 to bulge. Also as shown in FIGS. 2D and 3D, the bulging of device 10 may create substantial contact area between permeable body 12 and the bone surface at treatment site 22. Continued introduction of the bone treatment material under pressure into interior cavity 14 of device 10 may cause some bone treatment material to permeate out, for instance along path 30, of device 10 through the fabric forming permeable body 12. With further introduction of the bone treatment material, the bone treatment material permeating from permeable body 12 may penetrate the crevices and voids within the vertebral body at treatment site 22.

The bone treatment material may continue to be introduced into interior cavity 14 of device 10 under pressure such that the bulging and/or expansion of device 10 may compact the bone at treatment site 22. Again, means well known in the art, such as fluoroscopy, imaging, etc., may be used for closely monitoring the progress and location of the bone treatment material within treatment site 22, for ensuring that the bone treatment material remains confined within treatment site 22.

As previously discussed, the permeating bone treatment material may substantially encase the fabric of permeable body 12 of device 10. Thus, the fabric forming permeable body 12 may become an integral part of the bone treatment material. When the bone treatment material cures, the permeable fabric may provide additional structural integrity and strength to the vertebral body.

After a sufficient amount of bone treatment material has been introduced into interior cavity 14 of device 10 and/or an acceptable amount of bone treatment material has permeated through permeable body 12 and/or penetrated the crevices and voids at treatment site 22, introduction of the bone treatment material into interior cavity 14 of device 10 is terminated. The amount of bone treatment material that is sufficient may be either predetermined or determined during the process. Next, the bone treatment material may be permitted to cure, after which the distal end of cannula 28 may be detached from port 17 of device 10, and cannula 28 removed from the patient's body.

Alternately, upon termination of the introduction of the bone treatment material into interior cavity 14 of device 10, cannula 28 may be detached from port 17 of device 10, and cannula 28 removed from the patient's body. Opening 16 of device 10 may be securely closed shut so as to inhibit, or minimize, leakage of the bone treatment material out of device 10 through opening 16. The bone treatment material may be permitted to cure in-situ within device 10. Delivery device 26 may also be extracted from the patient's body if it had not been previously removed.

The foregoing description pertaining to one or more embodiments of the present invention has been for illustration purposes only. It is not intended to limit the invention. Various additions, subtractions, and/or modifications are possible in view of the exemplary embodiments discussed hereinabove, without departing from the scope and intent of the present invention. Accordingly, it is the intent of the present invention to embrace any and all alternatives as falling within the scope of the claims, together with any and all equivalents thereof.

We claim:

1. A device for treating a vertebral body comprising a hollow bag formed from a permeable fabric, the hollow bag adapted to receive a bone treatment material and including at least two regions of different permeability.

2. The device of claim 1 wherein the permeable fabric comprises a three-dimensional weave pattern.

3. The device of claim 2 wherein the three-dimensional weave defines a plurality of fully interconnected interstitial spaces.

4. The device of claim 3 wherein adjacent interstitial spaces are offset from one another.

5. The device of claim 4 wherein a degree of offset between said adjacent interstitial spaces affects a density of said permeable fabric.

6. The device of claim 2 wherein a density of said three-dimensional weave pattern affects a permeability of said permeable fabric.

7. The device of claim 2 wherein a density of said three-dimensional weave pattern affects a cross-sectional area of a flow-path through said permeable fabric.

8. The device of claim 2 wherein a density of said three-dimensional weave pattern affects a flow-path through said permeable fabric.

9. The device of claim 2 wherein a density of said three-dimensional weave pattern affects an expansion rate of said permeable fabric.

10. The device of claim 9 wherein said hollow bag comprises a first expansion region and a second expansion region, wherein an expansion rate of said first expansion region is different than an expansion rate of said second expansion region.

11. The device of claim 2 wherein a density of said three-dimensional weave pattern affects an elasticity of said permeable fabric.

12. The device of claim 11 wherein said hollow bag comprises a first elastic region and a second elastic region, wherein an elasticity of said first elastic region is different than an elasticity of said second elastic region.

13. The device of claim 2 wherein said hollow bag further comprises a first region and a second region, wherein a density of said three-dimensional weave pattern in said first region is different from a density of said three-dimensional weave pattern in said second region.

14. The device of claim 2 wherein said three-dimensional weave pattern includes a honeycomb weave pattern.

15. The device of claim 2 wherein the hollow bag is woven as one single element.

16. The device of claim 2 wherein said hollow bag further comprises at least one opening adapted for introducing said bone treatment material into said hollow bag.

17. The device of claim 16 wherein said hollow bag further comprises:
a port providing fluid communication between the at least one opening and an interior cavity of said hollow bag; and
a neck where said port securely attaches to said hollow bag.

18. The device of claim 17 wherein a permeability of said permeable fabric comprising said port is different from a permeability of said permeable fabric comprising said neck.

19. The device of claim 17 wherein a permeability of said permeable fabric comprising said hollow bag is different from a permeability of said permeable fabric comprising said neck.

20. The device of claim 17 wherein a permeability of said permeable fabric comprising said hollow bag is different from a permeability of said permeable fabric comprising said port.

21. The device of claim 17 wherein said port further comprises a back-flow restrictor.

22. The device of claim 21 wherein a distal end of said port includes said back-flow restrictor.

23. The device of claim 21 wherein said back-flow restrictor impedes outflow of the bone treatment material from said port.

24. The device of claim 16 wherein the at least one opening further includes a back-flow restrictor.

25. The device of claim 24 wherein said back-flow restrictor impedes outflow of the bone treatment material from the at least one opening in said hollow bag.

26. The device of claim 2 wherein said hollow bag is flexible.

27. The device of claim 2 further comprising a material applied to said permeable fabric in a desired pattern whereby the material affects the permeability of the fabric.

28. A device for treating a vertebral body comprising:
a hollow bag formed from a permeable fabric, said permeable fabric comprising a three-dimensional weave; and
at least one opening adapted for introducing a bone treatment material into said hollow bag.

29. The device of claim 28 wherein said three-dimensional weave includes fully interconnected interstitial spaces.

30. A method of treating a vertebral body, said method comprising the steps of:
inserting a device comprising a hollow bag formed from a permeable fabric, the hollow bag adapted to receive a bone treatment material and including one or more regions of predetermined and distinct permeability; and
introducing said bone treatment material under pressure into said device.

31. The method of claim 30 further comprising the step of inducing said bone treatment material to permeate through the permeable fabric of said device.

32. The method of claim 31 further including the step of inducing the permeated bone treatment material to penetrate voids and fissures in the vertebral body.

33. The method of claim 30 further comprising the step of applying a material to said permeable fabric in a desired pattern whereby the material effects the permeability of the fabric.
